(19) United States
(12) Patent Application Publication
(10) Pub. No.: US 2023/0012579 A1
(43) Pub. Date: Jan. 19, 2023

(54) Natural immunomodulator with antiviral activity

(71) Applicants: Elena Drobova, Novosibirsk (RU); Evgeny Tkachenko, Wuerzburg (DE)

(72) Inventors: Elena Drobova, Novosibirsk (RU); Evgeny Tkachenko, Wuerzburg (DE)

(21) Appl. No.: 17/374,584

(22) Filed: Jul. 13, 2021

(51) Int. Cl. A61K 31/713; A61K 36/064

(52) U.S. Cl. CPC A61K 31/713; A61K 36/064

(57) ABSTRACT

The present invention relates to methods for producing a mix of natural high polymeric and double stranded RNA from yeast (like Saccharomyces Cerevisiae), which can be used as dietary supplement, veterinary drug and medicine. The claimed invention relates to biotechnology and immunology, and to the production of the novel drug with a wide spectrum of antiviral activity on the basis RNA from yeast (like Saccharomyces Cerevisiae) for the treatment and prevention of diseases of viral etiology. Also, as a concomitant medication for the inflammatory diseases associated with bacterial infections. Our RNA works inside its own cell and activates a whole cascade of cellular and humoral immunity and therefore can be used as a polyvalent viral vaccine. By-products are high-purity proteins, low polymer RNA, microRNAs and oligonucleotides.

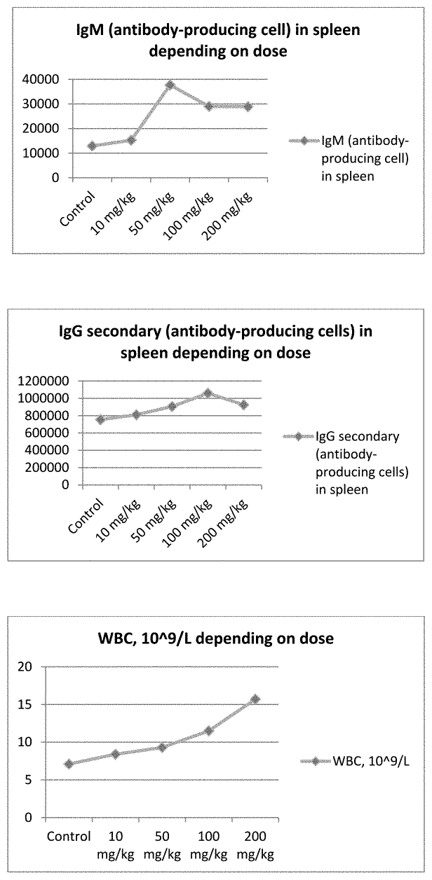

### BACKGROUND OF THE INVENTION

The immunostimulating properties of high-polymer RNA began to be studied in 1960ies. However the quality of this RNA was either significantly lower (short chains, individual nucleotides, ribo proteoglycan impurities, etc.) or contained highly toxic impurities due to aggressive detergents (lithium salts, SDS, 2-ethyl-hexanoic acid, etc.). Because the cell membrane contains chitin, it's hard to lyse it without damaging RNA inside the cell.

This high-polymer RNA is highly active-immunostimulating: it increases the synthesis of endogenous interferon by 9 times within just 3-6 hours after the administration. It also induces a leukocyte reaction, stimulates the primary (IgM) and secondary (IgG) humoral immune response (both—dose-dependently), and stimulates specific and non-specific immune response. Also, these RNA substances are active like polyclonal stimulants, regulating both the migration of T-lymphocytes and their cooperation with B-lymphocytes, and increasing the functional activity of macrophages and also they can be used like a polyvalent virus vaccine.

### BRIEF DESCRIPTION OF DRAWINGS

Graphs display the dose-response relationship of primary and secondary immune response and leukocyte reaction (in vivo studies with mice). In the Control group water was used for injection.

### DETAILED DESCRIPTION OF THE INVENTION

Yeasts slurring in the 1-3% aqueous solution of orthophosphoric acid titrated with alkali up to pH 7-8, the suspension should be heated up to 60-90 C during 3-20 min (conditions depends on yeast). Then an 2M NaOH solution is added to the resultant up to pH 8.0-8.2 and continue to heat this suspension with actively mixing during 20-30 minutes. At the next stage lysate stands for a 24 hours at room temperature (or centrifugate warm lysate), then the supernatant should be separated from a precipitate accurately and enzymes can be added (optionally). Then supernatants should be separated again. Add NaCl to obtain a 2.8-3.2M salt solution. Then leave for 20-24 hours at room temperature and centrifugate.

Produced RNA molecules should be washed by 2 portions of 3M NaCl solution and 3 portions of ethanol by resuspending at room temperature followed by a low-speed centrifugation. From the resulting precipitate the high polymeric RNA to be extracted with distilled water and dried with a conventional method, and ethanol is recovered by distillation. The invention makes it possible to obtain a high-polymer RNA with a significantly higher molecular weight. Using orthophosphoric acid and specific conditions allows it to damage the chitin membrane and to get the high-polymer RNA. Because nucleotides joined through phosphate groups, and RNA is polyanion, our detergent doesn't hydrolyse RNA. Special conditions of the process allow the control of the reaction balance in the solution and move it if necessary. Also the use of the safe reagents (ethanol, orthophosphoric acid, sodium chloride, water) significantly reduces the time needed for the wash out process.

The invention claimed is:

1. A method of lysis of the yeast cell membrane for the extraction of both natural high-polymer and double-stranded RNA, which have the pronounced immunostimulating and antiviral properties, the process comprising of:

2. A process as claimed in claim 1 wherein conducting a lysis a yeast cell wall

3. A process as claimed in claim 1 wherein using a phosphoric acid for the lysis of the yeast cell membrane

4. A process as claimed in claim 1 wherein using a low temperature to obtain high-polymer RNA from yeast

5. A process as claimed in claim 1 wherein using non-toxic compounds to obtain high-polymer RNA from yeast

6. A process as claimed in claim 1 wherein using enzymes to obtain RNA from yeast

7. A process as claimed in claim 1 wherein obtaining a natural high-polymer RNA from yeast

8. A process as claimed in claim 1 wherein obtaining a natural double-stranded RNA from yeast

9. Using biological natural high-polymer RNA as a drug, veterinary drug and food supplement, the process comprising of:

10. A process as claimed in claim 9 wherein using as a vaccine

11. A process as claimed in claim 9 wherein using as a hematopoiesis stimulator

12. A process as claimed in claim 9 wherein using as a substance which stimulates immune system

13. A process as claimed in claim 9 wherein using high-polimer natural RNA as antiviral agent

14. A process as claimed in claim 13, wherein, using as a substance which induces synthesis of an endogenous interferon.
